(19) United States
(12) Patent Application Publication
(10) Pub. No.: US 2004/0072307 A1
(43) Pub. Date: Apr. 15, 2004

(54) Processes for enhanced production of pantothenate

(76) Inventors: Christine Beck, Max-Joseph-Str. 35 Mannheim (DE); Hans-Peter Harz, Dudenhofen (DE)

Correspondence Address: LAHIVE & COCKFIELD, LLP., 28 STATE STREET, BOSTON, MA, 02109, US

(21) Appl. No.: 10/468,879

(22) PCT Filed: Mar. 11, 2002
(86) PCT No.: PCT/IB02/01982

(51) Int. Cl. C12P 13/04; C07C 229/04

(52) U.S. Cl. 435/106; 562/553

(57) ABSTRACT

The present invention features improved methods for producing pantothenate compositions. In particular, the invention features methods of culturing microorganisms such that spray-dryable compositions of pantothenate are produced. Also featured are pantothenate compositions produced by the processes herein-described.

### RELATED APPLICATIONS

The present application claims the benefit of prior-filed provisional Patent Application Serial No. 60/274,455, filed Mar. 9, 2001 (pending). The present invention is related to U.S. patent application Ser. No. 09/667,569, filed Sep. 21, 2000 (pending), which is a continuation-in-part of U.S. patent application Ser. No. 09/400,494, filed Sep. 21, 1999 (abandoned). U.S. patent application Ser. No. 09/667,569 also claims the benefit of prior-filed provisional Patent Application Serial No. 60/210,072, filed Jun. 7, 2000 (expired), provisional Patent Application Serial No. 60/221,836, filed Jul. 28, 2000 (expired), and provisional Patent Application Serial No. 60/227,860, filed Aug. 24, 2000 (expired). The entire contents of each of the above-referenced applications is incorporated herein by this reference.

### BACKGROUND OF THE INVENTION

D-pantothenic acid is produced on a large scale world wide. A large amount of the synthesized D-pantothenic acid is used as a feed additive for example in poultry and swine. The demand on D-pantothenic acid is increasing.

Pantothenate, also known as pantothenic acid or vitamin B5, is a member of the B complex of vitamins and is a nutritional requirement for mammals, including livestock and humans (e.g., from food sources, as a water soluble vitamin supplement or as a feed additive). In cells, pantothenate is used primarily for the biosynthesis of coenzyme A (CoA) and acyl carrier protein (ACP). These coenzymes function in the metabolism of acyl moieties which form thioesters with the sulfhydryl group of the 4′-phosphopantetheine portion of these molecules. These coenzymes are essential in all cells, participating in over 100 different intermediary reactions in cellular metabolism.

The conventional means of synthesizing pantothenate (in particular, the bioactive D isomer) is via chemical synthesis from bulk chemicals, a process which is hampered by excessive substrate cost as well as the-requirement for optical resolution of racemic intermediates. Accordingly, researchers have recently looked to bacterial or microbial systems that produce enzymes useful in pantothenate biosynthesis processes (as bacteria are themselves capable of synthesizing pantothenate). In particular, bioconversion processes have been evaluated as a means of favoring production of preferred isomer of pantothenic acid. Moreover, methods of direct microbial synthesis have recently been examined as a means of facilitating D-pantothenate production.

There is still, however, significant need for improved pantothenate production processes, in particular, for microbial processes optimized to produce higher yields of product and a more easily purified product.

### SUMMARY OF THE INVENTION

The present invention related to improved methods of producing pantothenate, in particular, methods of producing Ca-D-pantothenate containing compositions. The invention also features methods of producing spray-dryable pantothenate compositions, preferably spray-dryable compositions that include Ca-D-pantothenate. Ca-D-pantothenate containing compositions and/or spray-dryable pantothenate compositions of the present invention can be produced by fermentation of pantothenate-producing microorganisms from glucose by feeding Ca-salts during the course of the fermentation, preferably by feeding Ca-salts during the course of a pH-controlled fermentation. In a preferred embodiment, Ca-D-pantothenate containing compositions and/or spray-dryable pantothenate compositions are produced by the feeding of Ca(OH)2 during the course of the fermentation. Ca-D-pantothenate containing compositions and/or spray-dryable pantothenate compositions of the present invention can be produced by fermentation of pantothenate-producing microorganisms, preferably, microorganisms which have been engineered to produce pantothenate in a precursor independent manner. For example, Ca-D-pantothenate containing compositions and/or spray-dryable pantothenate compositions can be produced by fermentation of microorganisms engineered to produce pantothenate in a manner without the need for precursors such as β-alanine or pantoic acid (or pantoate). Also featured are methods of producing Mg-D-pantothenate compositions and/or spray-dryable compositions that include Mg-D-pantothenate, for example, methods that involve the feeding of Mg salts, preferably in a pH-controlled fermentation, most preferably the feeding of Mg(OH)₂. Ca-D-pantothenate containing composition, Mg-D-pantothenate compositions and/or spray-dryable compositions are prepared from the fermentation broth. The pantothenate compositions produced by the methods of the invention are powders (or compositions capable of being processed into powders), which contain salts of pantothenate, preferably divalent salts of pantothenate, and more preferably Ca-D-pantothenate or Mg-D-pantothenate. These production processes are more economical and efficient than conventional processes. The resulting products has many commercial uses, in particular, use as a vitamin source or as a feed additive.

The invention also pertains, at least in part, to a method for producing a spray-dryable pantothenate composition that includes culturing a pantothenate producing microorganism under Ca(OH)₂-controlled pH conditions, such that a spray-dryable pantothenate composition is produced. The method may further comprise spray drying the spray-dryable composition. Preferably, the spray-dryable pantothenate composition comprises Ca-D-pantothenate. The invention also pertains to the pantothenate compositions produced by the methods of the invention.

Other features and advantages of the invention will be apparent from the following detailed description and claims.

### DETAILED DESCRIPTION OF THE INVENTION

The invention pertains, at least in part, to a method of producing Ca-D-pantothenate, preferably a method for producing a spray-dryable Ca-D-pantothenate composition. The method includes culturing a pantothenate producing microorganism in the presence of Ca-salts, preferably in the presence of Ca-salts under controlled pH conditions, even more preferably under Ca(OH)₂-controlled pH conditions, such that Ca-D-pantothenate or a spray-dryable Ca-D-pantothenate composition is produced. The invention also pertains, at least in part, to a method of producing Mg-D-pantothenate, preferably a method for producing a spray-dryable Mg-D-pantothenate composition. The method includes culturing a pantothenate producing microorganism in the presence of Mg-salts, preferably in the presence of Mg-salts under controlled pH conditions, even more preferably under Mg(OH)₂-controlled pH conditions, such that Mg-D-pantothenate or a spray-dryable Mg-D-pantothenate composition is produced. The method may further comprise spray drying the spray-dryable composition. The pantothenate producing microorganisms may be cultured, for example, in fermentation medium or broth having compositions as defined herein. Preferably, the methods feature culturing recombinant pantothenate-producing microorganisms which have been engineered to produce pantothenate (e.g., to produce significant titers of pantothenate) in a manner independent of precursor feed.

So that the invention may be more readily understood, certain terms are first defined.

The term “pantothenate” includes the free acid form of pantothenate, also referred to as “pantothenic acid” as well as any salt thereof (e.g., derived by replacing the acidic hydrogen of pantothenate or pantothenic acid with a cation, for example, calcium, sodium, potassium, ammonium), also referred to as a “pantothenate salt”. Preferred pantothenate salts are calcium pantothenate, sodium pantothenate, magnesium pantothenate, potassium pantothenate and/or ammonium pantothenate. Pantothenate salts of the present invention include salts prepared via conventional methods from the free acids described herein. In another embodiment, a pantothenate salt is synthesized directly by a microorganism of the present invention. A pantothenate salt of the present invention can likewise be converted to a free acid form of pantothenate or pantothenic acid by conventional methodology. A preferred pantothenate salt is Ca-D-pantothenate (ie., Ca(D-pantothenate)₂). Another preferred pantothenate salt is Mg-D-pantothenate (i.e., Mg(D-pantothenate)₂). Art-recognized methods of producing Ca-D-pantothenate include producing Ca-D-pantothenate from D-pantothenic acid by adding equimolar amounts of Ca(OH)₂. D-pantothenate is routinely isolated from fermentation medium or broth containing D-pantothenate by methods including, but not limited to, those described in WO 96/33283, U.S. Pat. No. 6,013,492 and DE 10016321.

D-pantothenate can be produced by fermentation of a microorganism in a broth containing a carbon source such as sugars (e.g. glucose, sucrose, molasses) or other carbohydrates (e.g. starch hydrolysates), precursors such as β-Alanine, pantoic acid (or pantoate), ketopantoate (or ketopantoic acid), α-ketoisovalerate (or α-ketoisovaleric acid) and the like, nitrogen sources such as (NH₄)₂SO₄, protein sources such as soy flour, corn steep liquor or yeast extract, phosphor sources such as potassium or sodium phosphates and trace minerals and vitamins. The microorganism is grown in the fermentation broth at a suitable pH, with an appropriate stirrer and air flow rate.

The term “pantothenate composition” refers to compositions which include pantothenate and, optionally, additional components including, but not limited to, buffers, salts, and/or other media components, media remnants (i e., remnants of complex media components from the fermentation broth), biomass (e.g., microorganisms and/or portions or remnants of microorganisms from the fermentation broth), and/or media components which aid in the formulation of the product (such as sugars, products from cereals or legumes, silica gel etc.)

The term “spray-dryable pantothenate composition” includes pantothenate compositions from which liquid components can be evaporated or other wise removed to yield a solid composition. Advantageously, the spray-dryable pantothenate composition is spray dried or spray-granulated (e.g., using a fluidized bed spray dryer), although other methods of removing liquid components also may be used (e.g., evaporation, lyophilization, and the like). The spray-dryable pantothenate composition may be dried with or without separation from the biomass in the fermentation broth, e.g., by filtration, centrifugation, ultrafiltration, microfiltration, or combinations thereof. In an embodiment, the dried spray-dryable pantothenate composition is capable of performing its intended function without additional purification steps. For example, the dried spray-dryable pantothenate composition may be added directly to animal feed (e.g., feed for poultry or swine) or added to feed premixes without further purification procedures.

Examples of commercial spray dryer apparatus include those produced by Niro or APV Anhydro (both of Copenhagen, Denmark). Fluidized spray bed granulators are produced by Glatt (Bingen, Germany), Heinen (Varel, Germany), Niro-Aeromativ (Bubendorf, Switzerland) and Allgaier (Uhingen, Germany). In a preferred embodment, the inlet temperature in a spray dryer is set at about 100° C. to about 280° C., and advantageously, at about 120° C. to about 210° C. The outlet temperature in a spray dryer is set to about a range of 30° C. to about 180° C., advantageously at about 50° C. to about 150° C., and preferably from about 50° C. to about 100° C. The atomization of the liquid is done by a 2 fluid nozzle (pneumatic nozzle, pressure nozzle) or by a rotating disc. Also a FSD (Fluidized spray dryer) produced by Niro (Copenhagen, Denmark) or a comparable dryer called SBD (Spray bed dryer) produced by APV Anhydro (Copenhagen, Denmark) can be used for the drying. These dryers represent a combination of a spray dryer and a fluidized bed granulator. It is also possible to have a certain agglomeration during the drying. For the selection of a determined particle size distribution in the final product, very small particles can be separated by sieving and returned into the process. Likewise very large particles can be-crushed in a mill and returned into the process.

The term “pantothenate producing microorganism” includes naturally-occurring microorganisms which produce pantothenate as well as microorganisms, e.g., recombinant microorganisms, having a deregulated pantothenate biosynthetic pathway and/or a deregulated isoleucine-valine biosynthetic pathway. As used herein, a microorganism “having a deregulated pantothenate biosynthetic pathway” includes a microorganism having at least one pantothenate biosynthetic enzyme deregulated (e.g., overexpressed) such that pantothenate production is enhanced (e.g., as compared to pantothenate production in said microorganism prior to deregulation of said biosynthetic enzyme or as compared to a wild-type microorganism). Preferably, a microorganism “having a deregulated pantothenate biosynthetic pathway” includes a microorganism having at least one pantothenate biosynthetic enzyme deregulated (e.g., overexpressed) such that pantothenate production is 1 g/L or greater. More preferably, a microorganism “having a deregulated pantothenate biosynthetic pathway” includes a microorganism having at least one pantothenate biosynthetic enzyme deregulated (e.g., overexpressed) such that pantothenate production is 2 g/L or greater.

The term “pantothenate biosynthetic enzyme” includes any enzyme utilized in the formation of a compound (e.g., intermediate or product) of the pantothenate biosynthetic pathway. For example, synthesis of pantoate from α-ketoisovalerate (α-KIV) proceeds via the intermediate, ketopantoate. Formation of ketopantoate is catalyzed by the pantothenate biosynthetic enzyme ketopantoate hydroxymethyltransferase (the panB gene product). Formation of pantoate is catalyzed by the pantothenate biosynthetic enzyme ketopantoate reductase (the panE gene product). Synthesis of β-alanine from aspartate is catalyzed by the pantothenate biosynthetic enzyme aspartate-α-decarboxylase (the panD gene product). Formation of pantothenate from pantoate and β-alanine (e.g., condensation) is catalyzed by the pantothenate biosynthetic enzyme pantothenate synthetase (the panC gene product).

The term “isoleucine-valine biosynthetic pathway” includes the biosynthetic pathway involving isoleucine-valine biosynthetic enzymes (e.g. polypeptides encoded by biosynthetic enzyme-encoding genes), compounds (e.g., precursors, substrates, intermediates or products), cofactors and the like utilized in the formation or synthesis of conversion of pyruvate to valine or isoleucine. The term “isoleucine-valine biosynthetic pathway” includes the biosynthetic pathway leading to the synthesis of valine or isoleucine in a microorganisms (e.g., in vivo) as well as the biosynthetic pathway leading to the synthesis of valine or isoleucine in vitro.

The term “isoleucine-valine biosynthetic enzyme” includes any enzyme utilized in the formation of a compound (e.g., intermediate or product) of the isoleucine-valine biosynthetic pathway. Synthesis of valine from pyruvate proceeds via the intermediates, acetolactate, α,β-dihydroxyisovalerate (α,β-DHIV) and α-ketoisovalerate (α-KIV). Formation of acetolactate from pyruvate is catalyzed by the isoleucine-valine biosynthetic enzyme acetohydroxyacid synthetase (the ilvBN gene product, or alternatively, the alsS gene product). Formation of α,β-DHIV from acetolactate is catalyzed by the isoleucine-valine biosynthetic enzyme acetohydroxyacidisomero reductase (the ilvC gene product). Synthesis of α-KIV from α,β-DHIV is catalyzed by the isoleucine-valine biosynthetic enzyme dihydroxyacid dehydratase (the ilvD gene product). Moreover, valine and isoleucine can be interconverted by branched chain amino acid transaminases.

In one embodiment, a recombinant microorganism of the present invention is a Gram positive organism (e.g., a microorganism which retains basic dye, for example, crystal violet, due to the presence of a Gram-positive wall surrounding the microorganism). In a preferred embodiment, the recombinant microorganism is a microorganism belonging to a genus selected from the group consisting of Bacillus, Cornyebacterium, Lactobacillus, Lactococci and Streptomyces. In a more preferred embodiment, the recombinant microorganism is of the genus Bacillus. In another preferred embodiment, the recombinant microorganism is selected from the group consisting of Bacillus subtilis, Bacillus lentimorbus, Bacillus lentus, Bacillus firmus, Bacillus pantothenticus, Bacillus amyloliquefaciens, Bacillus cereus, Bacillus circulans, Bacillus coagulans, Bacillus licheniformis, Bacillus megaterium, Bacillus pumilus, Bacillus thuringiensis, and other Group 1 Bacillus species, for example, as characterized by 16 S rRNA type (Priest (1993) in Bacillus subtilis and Other Gram-Positive Bacteria eds. Sonenshein et al., ASM, Washington, D.C., p. 6). In another preferred-embodiment, the recombinant microorganism is Bacillus-brevis or Bacillus stearothermophilus. In another preferred embodiment, the recombinant microorganism is selected from the group consisting of Bacillus licheniformis, Bacillus amyloliquefaciens, Bacillus halodurans, Bacillus subtilis, and Bacillus pumilus.

In another embodiment, the recombinant microorganism is a Gram negative (excludes basic dye) organism. In a preferred embodiment, the recombinant microorganism is a microorganism belonging to a genus selected from the group consisting of Salmonella, Escherichia, Klebsiella, Serratia, and Proteus. In a more preferred embodiment, the recombinant microorganism is of the genus Escherichia. In an even more preferred embodiment, the recombinant microorganism is Escherichia coli. In another embodiment, the recombinant microorganism is Saccharomyces (e.g., S. cerevisiae). Particularly preferred “pantothenate producing microorganisms” include those described, for example, in U.S. patent applciation Ser. No. 09/667,569.

The term “culturing” includes maintaining and/or growing a living microorganism of the present invention (e.g., maintaining and/or growing a culture or strain). In one embodiment, a microorganism of the invention is cultured in liquid media, e.g., fermentation broth. In a preferred embodiment, a microorganism of the invention is cultured in media (e.g., a sterile, liquid medium) comprising nutrients essential or beneficial to the maintenance and/or growth of the microorganism (e.g., carbon sources or carbon substrate, for example carbohydrate, hydrocarbons, oils, fats, fatty acids, organic acids, and alcohols; nitrogen sources, for example, peptone, yeast extracts, meat extracts, malt extracts, urea, ammonium sulfate, ammonium chloride, ammonium nitrate and ammonium phosphate; phosphorus sources, for example, phosphoric acid, sodium and potassium salts thereof; trace elements, for example, magnesium, iron, manganese, calcium, copper, zinc, boron, molybdenum, and/or cobalt salts; as well as growth factors such as amino acids, vitamins, growth promoters and the like).

Preferably, microorganisms of the present invention are cultured under controlled pH conditions. In one embodiment, microorganisms are cultured at a pH of between 6.0 and 11.0. In another embodiment, the microorganisms are cultured at a pH of between 6.0 and 8.5, e.g., at a pH of about 7. Preferred reagents for controlling pH include ammonia hydroxide, sodium hydroxide and/or potassium hydroxide. Use of such reagents to control pH is particularly important when salts (e.g., divalent cations, for example, Ca²⁺ (CaCl₂) or Mg²⁺ (MgCl₂) are added to the fermentation media). In a prefered embodiment, the microorganisms are cultured under “Ca(OH)₂-controlled pH conditions”. The term “Ca(OH)₂-controlled pH conditions” includes conditions to which at least some Ca(OH)₂ has been added, advantageously, to yield a desired product, e.g., a spray-dryable Ca-pantothenate composition. Preferably, the desired pH is maintained by adding Ca(OH)₂, when necessary, to raise the pH, and by lowering the pH by any methods known to those skilled in the art, when necessary. In another prefered embodiment, the microorganisms are cultured under “Mg(OH)₂-controlled pH conditions”. The term “Mg(OH)₂-controlled pH conditions” includes conditions to which at least some Mg(OH)₂ has been added, advantageously, to yield adesired product, e.g., a spray-dryable Mg-pantothenate composition. Preferably, the desired pH is maintained by adding Mg(OH)₂, when necessary, to raise the pH, and by lowering the pH by any methods known to those skilled in the art, when necessary.

Microorganisms are cultured under conditions such that at least 20 g/L of pantothenate are produced in about 36 hours, at least about 20-30 g/L are produced in about 48 hours or at least about 35 to 40 g/L are produced in about 72 hours. By media, process or strain optimization or by the combination of the three the concentration of pantothenate in the final broth can reach 40 g/L, 45 g/L, 50 g/L, 55 g/L, 60 g/L, 65 g/L, 70 g/L, 80 g/L, 90 g/L or even more than 90 g/L.

Calcium-D-pantothenate is widely used as a feed additive. Ca-D-pantothenate is also found as an ingredient of “pre-mixes”. “Pre-mixes” are art-recognized compositions (e.g., feed additives) that include, for example, vitamins, minerals and/or amino acids which support animal growth and/or health. It is therefore highly desirable to design a process in which Ca-D-pantothenate is produced from a renewable source such as sugar without need-to add any pantothenate-precursors, e.g., β-alanine.

For this purpose Ca-ions can be added to the fermentation broth containing D-pantothenic acid or its salts after the end of the fermentation at any step of the down stream processing as described in patent application DE10046490. In another embodiment, Ca-ions can be added to the fermentation broth during the course of the fermentation. For example Ca-ions can be added to the fermentation broth by feeding solutions containing CaO, Ca(OH)₂, CaCl₂, CaCO₃, CaSO₄, CaHPO₄ or organic Ca-salts such as Ca-forniate, Ca-acetate, Ca-propionate, Ca-glycinate or Ca-lactate or a combination of these salts. Also other Ca-salts can be used; this enumeration shall not be regarded as limiting. Preferably CaO or Ca(OH)₂ are used in the fermentation, because these compounds will help with titration of the pH. Preferably, at least 1 mole of Ca-salt is added for 2 moles of D-pantothenate produced. In another embodiment, greater than 1 mole of Ca-salt might be added for 2 moles of D-pantothenate produced. In another embodiment, additional Ca-salts as enumerated above are added to the fermentation broth generated by feeding a Ca-salt during the fermetnation process after the fermentation has ended. (see e.g., Examples 4 & 5).

The Ca-D-pantothenate containing fermentation broth can be spray dried or spray-granulated, as described herein. In one embodiment compounds such as sugars, e.g. lactose or maltodextrine, products from cereals or legumes, e.g. wholemeal, bran or flour from soy or wheat, mineral salts, e.g. Ca-, Mg-, Na- and K-salts, additives such as silica gel and also D-pantothenic acid and/or its salts (produced by chemical synthesis or fermentation) are added to the fermentation broth prior to or during the spray drying or spray granulating process.

In a preferred embodiment no additional components are added and the fermentation broth is spray dried directly.

In one embodiment the biomass is separated from the fermentation broth and only the supernatant is spray dried. Biomass separation is performed by techniques such as filtration, centrifugation, ultrafiltration, microfiltration or combinations thereof. The obtained biomass might be subjected to a washing step, the liquid being added to the separated fermentation supernatant. In another embodiment the biomass-containing fermentation broth is spray dried without separation of the biomass. In yet another embodiment the fermentation broth is spray dried without additional concentration step.

In another embodiment, concentration of the fermentation broth is performed. As a consequence the dry matter content is increased. This can, for example, be achieved by withdrawal of water by evaporation. Evaporation can be performed in multiple steps and under vacuum. The evaporation can be done on a thin film evaporater, as for example produced by the companies GIG (4800 Attnang Puchheim, Austria), GEA Canzler (52303 Düren, Germany), Diessel (31103 Hildesheim, Germany) and Pitton (35274 Kirchhain, Germany). The dry matter content in the fermentation broth can also be increased by the use of membrane techniques (e.g., nanofiltration, reverse osmosis, etc.). After concentration, the dry matter content may be from about 20% to about 80%. In an embodiment, the removed water is returned into the fermentation broth, reducing the amount of waste water produced.

In one embodiment sterilization of the fermentation broth is performed in the fermentor directly after the end of the fermentation. In another embodiment, sterilization is performed after the broth has left the fermentor. Also sterilization of the culture supernatant after removal of the biomass from the fermentation broth by means of separation as outlined above is possible.

The drying or formulation of the fermentation broth can be performed by conventional means as known in the art. For example spray drying, fluidized bed spray granulation or spin-flash drying of fermentation broth can be used (Ullmann's Encyclopedia of Industrial Chemistry, 6^{th} edition, 1999, electronic release, chapter “Drying of solid materials”).

The product obtained by the present invention can include in addition to Ca-D-pantothenate, other components of the fermentation broth, e.g. phosphates, carbonates, remaining carbohydrates, biomass, complex media components etc. The product characteristically has a white to brown color, a water content of less than 5%, preferably 1-3%. To prevent clotting of the product, a water content of 5% should not be exceeded. The content of Ca-D-pantothenate is 10-90%, preferably 20-80%, more preferably 50-80%.

Likewise Ca-D-pantothenate containing fermentation broth can be prepared from glucose with no need of feeding β-Alanine or any other pantothenate-precursor and reaching D-pantothenate titers of 20, 25, 30, 35, 40, 45, 50, 55, 60, 65, 70, 75, 80, 85, and more than 90 g/L. In a preferred embodiment, microorganisms of the present invention are cultured under controlled aeration. The term “controlled aeration” includes sufficient aeration (e.g., oxygen) to result in production of the desired product (e.g., spray-dryable pantothenate). In one embodiment, aeration is controlled by regulating oxygen levels in the culture, for example, by regulating the amount of oxygen dissolved in culture media. Preferably, aeration of the culture is controlled by agitating the culture. Agitation may be provided by a propeller or similar mechanical agitation equipment, by revolving or shaking the culture vessel (e.g., tube or flask) or by various pumping equipment. Aeration may be further controlled by the passage of sterile air or oxygen through the medium (e.g., through the fermentation mixture). Also preferably, microorganisms of the present invention are cultured without excess foaming (e.g., via addition of antifoaming agents).

Moreover, microorganisms of the present invention can be cultured under controlled temperatures. The term “controlled temperature” includes any temperature which results in production of the desired product (e.g., spray-dryable pantothenate). In one embodiment, controlled temperatures include temperatures between 15° C. and 95° C. In another embodiment, controlled temperatures include temperatures between 15° C. and 70° C. Preferred temperatures are between 20° C. and 55° C., more preferably between 30° C. and 50° C.

Microorganisms can be cultured (e.g., maintained and/or grown) in liquid media and preferably are cultured, either continuously or intermittently, by conventional culturing methods such as standing culture, test tube culture, shaking culture (e.g., rotary shaking culture, shake flask culture, etc.), aeration spinner culture, or fermentation. In a preferred embodiment, the microorganisms are cultured in shake flasks. In a more preferred embodiment, the microorganisms are cultured in a fermentor (e.g., a fermentation process). Fermentation processes of the present invention include, but are not limited to, batch, fed-batch and continuous processes or methods of fermentation. The phrase “batch process” or “batch fermentation” refers to a system in which the composition of media, nutrients, supplemental additives and the like is set at the beginning of the fermentation and not subject to alteration during the fermentation, however, attempts may be made to control such factors as pH and oxygen concentration to prevent excess media acidification and/or microorganism death. The phrase “fed-batch process” or “fed-batch” fermentation refers to a batch fermentation with the exception that one or more substrates or supplements are added (e.g., added in increments or continuously) as the fermentation progresses. The phrase “continuous process” or “continuous fermentation” refers to a system in which a defined fermentation media is added continuously to a fermentor and an equal amount of used or “conditioned” media is simultaneously removed, preferably for recovery of the desired product (e.g., a spray-dryable pantothenate composition). A variety of such processes have been developed and are well-known in the art.

In an embodiment, the spraydryable pantothenate composition is not purified from the microorganism, for example, when the microorganism is biologically non-hazardous (e.g., safe). For example, the entire culture or fermentation broth (or supernatant) can be used as a source of product (e.g., crude product). In one embodiment, the culture (or culture supernatant) is used without modification. In another embodiment, the culture (or culture supernatant) is concentrated. In yet another embodiment, the culture (or culture supernatant) is dried or lyophilized.

A production method of the present invention results in production of the desired compound at a significantly high yield. The phrase “significantly high yield” includes a level of production or yield which is sufficiently elevated or above what is usual for comparable production methods, for example, which is elevated to a level sufficient for commercial production of the desired product (e.g., production of the product at a commercially feasible cost). In one embodiment, the invention features a production method that includes culturing a recombinant microorganism under conditions such that the desired product (e.g., pantothenate) is produced at a level greater than 2 g/L. In another embodiment, the invention features a production method that includes culturing a recombinant microorganism under conditions such that the desired product (e.g., pantothenate) is produced at a level greater than 10 g/L. In another embodiment, the invention features a production method that includes culturing a recombinant microorganism under conditions such that the desired product (e.g., pantothenate) is produced at a level greater than 20 g/L. In yet another embodiment, the invention features a production method that includes culturing a recombinant microorganism under conditions such that the desired product (e.g., pantothenate) is produced at a level greater than 30 g/L. In yet another embodiment, the invention features a production method that includes culturing a recombinant microorganism under conditions such that the desired product (e.g., pantothenate) is produced at a level greater than 40 g/L. In yet another embodiment, the invention features a production method that includes culturing a recombinant microorganism under conditions such that the desired product (e.g., pantothenate) is produced at a level greater than 50 g/L. In yet another embodiment, the invention features a production method that includes culturing a recombinant microorganism under conditions such that the desired product (e.g., pantothenate) is produced at a level greater than 60 g/L. The invention further features a production method for producing the desired compound that involves culturing a recombinant microorganism under conditions such that a sufficiently elevated level of compound is produced within a commercially desirable period of time.

In yet another embodiment, the invention features a production method that includes culturing a recombinant organism under conditions such that the desired product (e.g., pantothenate) is produced, collecting the culture, separating the biomass from the broth (or not), sterilizing the culture (before or after biomass removal), concentrating the broth (or not) and drying the culture by any means described above such that a Ca-D-pantothenate is contained in the product at a level greater than 10% (20-30-40% etc).

This invention is further illustrated by the following examples which should not be construed as limiting. The contents of all references, patents and published patent applications cited throughout this application are-incorporated herein by reference.

### EXEMPLIFICATION OF THE INVENTION

### EXAMPLE 1

### Calcium-Pantothenate Production with Strain PA668-24

In a 20 L lab scale fermentor (Infors AG, Switzerland) 4 liters of a water based fermentation batch medium was prepared according to the following table:

| Material | Final Concentration | |
|---|---|---|
| Soy Flour | 40 | g/L |
| Yeast Extract | 5 | g/L |
| Na Glutamate | 5 | g/L |
| (NH₄)₂SO₄ | 8 | g/L |
| Tego KS 911 (antifoam) | 1 | mL/L |

Water was added to 4 L final volume. After sterilization (121° C., 30 min) 1 liter of a sterile solution was added. The concentrations of the broth components are as follows:

| Material | Concentration | |
|---|---|---|
| KH₂PO₄ | 10 | g/L |
| K₂HPO₄ | 20 | g/L |
| Glucose | 20 | g/L |
| CaCl₂ | 0.1 | g/L |
| MgCl₂ | 1 | g/L |
| Na Citrate | 1 | g/L |
| FeSO₄ × 7 H₂O | 0.01 | g/L |
| SM-1000x | 1 | mL/L |

The trace mineral solution SM-1000× had following composition: 0,15 g Na₂MoO₄×2 H₂O, 2,5 g H₃BO₃, 0,7 g CoCl₂×6 H₂O, 0,25 g CuSO₄×5 H₂O, 1,6 g MnCl₂×4 H₂O, 0,3 g ZnSO₄×7 H₂O were dissolved in water and filled up to 1 liter. SM-1000× was added via a sterile syringe to the fermentation batch medium.

To a starting volume of 5 liters, 100 mL of an inoculum culture (OD=10 in SVY medium) of Bacillus subtilis strain PA668-24 was added to the batch medium.

The inoculum was prepared by inoculating 100 mL of SVY medium with a cryo stock of strain PA668-24 supplemented with 15 mg/L tetracycline and 5 mg/L chloramphenicol. The SVY medium made from a sterilized mixture of 25 g of Difco Veal Infusion broth, 5 g of Difco Yeast extract, 5 g of Na glutamate, and 2.7 g (NH₄)₂SO₄ in 740 mL water. To the sterilized medium, 200 mL of sterile 1 M K₂HPO₄ (pH 7) and 60 mL of sterile 50% Glucose-solution was added to yield a final volume of one liter. The culture was then incubated at 37° C. for 12-18 hours on a rotary shaker.

The cryo stock was prepared in a 250 mL Erlenmeyer flask with baffles. 50 mL of SVY-Medium was supplemented with 15 mg/L of tetracycline and 5 mg/L of chloramphenicol and inoculated with strain PA668-24 from a single colony on an agar plate. After incubation on a rotary shaker over night, 10 mL of sterile 80% glycerol solution was added to the culture. Aliquots of 1 mL were prepared in cryo tubes an frozen individually at −80° C.

After inoculation, the fermentation was started. The temperature was set at 43° C. The initial stirrer speed was set at 400 rpm and the initial air flow rate was set at 4 L/min. All fermentations were glucose-limited fed batch processes. The initial batched 2% glucose was consumed during exponential growth. Afterwards, glucose concentrations were maintained between 0 and 1 g/L by continuous feeding of a glucose solution as outlined in following table:

| Material | Final Concentration | |
|---|---|---|
| Glucose | 600 | g/L |
| Na Glutamate | 5 | g/L |
| Na Citrate | 2 | g/L |
| FeSO₄ × 7 H₂O | 0.02 | g/L |
| SM-1000x | 2 | mL/L |

During the first 8 hours of the fermentation, the pH was maintained the addition of a 25% NH₃ solution. Subsequently, the pH was controlled by adding a 25% aqueous suspension of Ca(OH)₂ to the fermentation broth to raise the pH when necessary. Occasionally, when the pH was above preferred pH range, it was lowered by the addition of 20% phosphoric acid. The stirrer speed and the air flow rate were controlled by the dissolved oxygen value (pO₂), which was set at 20% of the saturation value. The feeding of glucose solution was controlled by an algorithm linked to the pO₂ value. To control the foaming, an antifoam agent was added occasionally. At 48 hours fermentation time, the feeding with glucose solution was stopped.

After the pO₂ had reached a value of 95%, the fermentation broth was collected. The D-Pantothenate concentration was 21.4 g/L. The biomass was separated by centrifugation. The cells remaining in the supernatant were killed by sterilization at 121° C. for 30 minutes, which was proven by plating a sample of the broth on an agar plate (Difco Tryptone Blood Agar Broth, 33 g/L, supplemented with 30 mg/L tetracycline and 30 mg/L chloramphenicol), and incubating it over night at 37° C. and checking for colony growth. The concentration of D-pantothenate in the final supernatant was 15 g/L.

The fermentation broth was concentrated in a thin film evaporator yielding a final dry mass content of 21%. The concentrated fermentation broth contained 55.4 g/L Ca-D-pantothenate.

### EXAMPLE 2

### Formulated Fermentation Broth Containing Ca-D-Pantothenate

Five hundred grams of the concentrated fermentation broth generated in Example 1 was dried on a lab scale spray dryer with a two-fluid fountain nozzle, diameter 1.2 mm (Minor ‘Hi-Tec’; Niro, Copenhagen, Denmark). The homogeneity-of the fermentation broth suspension was maintained by continuous stirring. The inlet temperature was 185-192° C., the outlet temperature was 88-91° C., and the air pressure was 2 bar.

74.8 g of a yellow-brownish powder was obtained, for a yield of 70%. The powder contained 25% Ca-D-pantothenate. The moisture content was 1.7%.

### EXAMPLE 3

### Formulated Fermentation Broth Containing Ca-D-Pantothenate

Five hundred grams of the concentrated fermentation broth generated in Example 1 was dried on a lab scale spray dryer with a two-fluid fountain nozzle, diameter 1.2 mm (Minor ‘Hi-Tec’; Niro, Copenhagen, Denmark). The homogeneity of the fermentation broth suspension was maintained by continuous stirring. The inlet temperature was 153-159° C., the outlet temperature was 72-78° C., and the air pressure was 2 bar.

79.2 g of a yellow-brownish powder was obtained. The powder contained 24.1% Ca-D-pantothenate. The moisture content was 1.9%.

### EXAMPLE 4

### Formulated Fermentation Broth Containing Ca-D-Pantothenate

To 500 g of the concentrated fermentation broth generated in Example 1, 2.2 g of solid Ca(OH)₂ was added to increase the basicity of the solution to pH 10. The solution was then dried on a lab scale spray dryer with a two-fountain nozzle, diameter 1.2 mm (Minor ‘Hi-Tec’; Niro, Copenhagen, Denmark). The homogeneity of the fermentation broth suspension was maintained by continuous stirring. The inlet temperature was 135-143° C., and the outlet temperature was 73-77° C. The air pressure was 2 bar.

82.4 g of a yellow-brownish powder was obtained. The powder contained 23.7% Ca-D-pantothenate. The moisture content was 1.6%.

### EXAMPLE 5

### Formulated Fermentation Broth Containing Ca-D-Pantothenate

To 500 g of the concentrated fermentation broth generated in Example 1, 5.0 g solid CaCl₂ was added. The obtained solution was dried on a lab scale spray dryer with a two-fluid fountain nozzle, diameter 1.2 mm (Minor ‘Hi-Tec’; Niro, Copenhagen, Denmark). The homogeneity of the fermentation broth suspension was maintained by continuous stirring. The inlet temperature was 129-130° C. and the outlet temperature was 75-78° C. The air pressure was 2 bar.

65.1 g of a yellow-brownish powder was obtained. The powder contained 23.4% Ca-D-pantothenate. The moisture content was 1.7%.

### EXAMPLE 6

### Calcium-Pantothenate Production with Strain PA668-2A

In a 20 L lab scale fermentor (Infors AG, Switzerland), four liters of a water based fermentation batch medium is prepared according to the following table:

| Material | Final Concentration | |
|---|---|---|
| Soy Flour | 40 | g/L |
| Yeast Extract | 5 | g/L |
| Na Glutamate | 5 | g/L |
| (NH₄)₂SO₄ | 8 | g/L |
| Tego KS (antifoam) | 1 | mL/L |

Water is added to yield a 4 L final volume. After sterilization at 121° C. for 30 minutes, one liter of a sterile solution is added to reach the final concentrations shown in the following table:

| Material | Final Concentration | |
|---|---|---|
| KH₂PO₄ | 10 | g/L |
| K₂HPO₄ | 20 | g/L |
| Glucose | 20 | g/L |
| CaCl₂ | 0.1 | g/L |
| MgCl₂ | 1 | g/L |
| Na Citrate | 1 | g/L |
| FeSO₄ × 7 H₂O | 0.01 | g/L |
| SM-1000x | 1 | mL/L |

The trace mineral solution SM-1000× is comprised of a combination of 0.15 g Na₂MoO₄×2 H₂O, 2.5 g H₃BO₃, 0.7 g CoCl₂×6 H₂O, 0.25 g CuSO₄×5 H₂O, 1.6 g MnCl₂×4 H₂O, and 0.3 g ZnSO₄×7 H₂O dissolved in one liter of water. The trace mineral solution, SM-1000×, is added via a sterile syringe to the fermentation batch medium.

The starting volume of the fermentation batch medium is five liters. 100 mL of an inoculum culture (OD=10 in SVY medium) of Bacillus subtilis-strain (PA668-2A) is added to the batch medium.

To prepare the inoculum, 100 mL of SVY medium (supplemented with 15 mg/L Tetracycline and 5 mg/L Chloramphenicol) is inoculated with a cryo stock of strain, PA668-2A. SVY medium: Difco Veal Infusion broth 25 g, Difco Yeast extract 5 g, Na Glutamate 5 g, (NH₄)₂SO₄ 2.7 g in 740 mL H₂O, sterilize; add 200 mL of sterile 1M K₂HPO₄ (pH 7) and 60 mL of sterile 50% Glucose-solution (final volume 1 L)). The culture was incubated at 37° C. for 12-18 hours on a rotary shaker.

The cryo stock is prepared in a 250 mL Erlenmeyer flask with baffles. 100 mL of SVY-Medium (supplemented with 15 mg/L of tetracycline and 5 mg/L of chloramphenicol) is inoculated with strain PA668-2A from a single colony on an agar plate. After incubation on a rotary shaker overnight, 10 mL of sterile 80% glycerol solution is added to the culture. Culture aliquots of 1 mL are prepared in cryo tubes an frozen individually at −80° C.

After inoculation, the fermentation is started. The temperature is set at 43° C., the initial stirrer speed is set at 400 rpm, and the air flow rate is set at 4 L/min.

All fermentations are glucose-limited fed batch processes. The initial batched 2% Glucose is consumed during exponential growth. Afterwards, glucose concentrations are maintained between 0 and 1 g/L by continuous feeding of a 800 g/L glucose solution as outlined in following table:

| Material | Final Concentration | |
|---|---|---|
| Glucose | 800 | g/L |
| CaCl₂ | 0.6 | g/L |
| Na Glutamate | 5 | g/L |
| Na Citrate | 2 | g/L |
| FeSO₄ × 7 H₂O | 0.02 | g/L |
| SM-1000x | 2 | mL/L |

During the first 24 hours of the fermentation, the pH is controlled by adding a 25% NH₃ solution. After that, the pH is controlled by adding a 25% aqueous suspension of Ca(OH)₂ to the fermentation broth. For titration of rarely occurring basic pH, 20% phosphoric acid is added. The stirrer speed and the air flow are controlled by the dissolved oxygen value (pO₂), which is set at 20% of the saturation value. The feeding of glucose solution is controlled by an algorithm linked to the pO₂ value. The foaming is controlled by occasionally adding an antifoam agent. At 48 hours fermentation time, the feeding with glucose solution is stopped. The D-pantothenate concentration is about 44.8 g/L. After the pO₂ has reached, 95% the fermentation broth is sterilized at 121° C. for 30 min. The successful sterilization can be proven by plating a sample of the broth on an agar plate (Difco Tryptone Blood Agar Broth 33 g/L supplemented with 30 mg/L tetracycline and 30 g/L chloramphenicol), incubating it over night at 37° C., and then checking it for colony growth. The biomass is not removed from the broth which contains 38 g/L D-Pantothenate.

The broth is concentrated on a thin film evaporator to reach a final dry mass content of 30%.

### EXAMPLE 7

### Formulated Fermentation Broth Containing Ca-D-Pantothenate and Biomass

500 g of the concentrated fermentation broth generated in example 6 is dried on a lab scale spray dryer with a two-fluid fountain nozzle, diameter 1.2 mm (Minor ‘Hi-Tec’; Niro, Copenhagen, Denmark). The homogeneity of the fermentation broth suspension was maintained by continuous stirring. The inlet temperature is 185-192° C. and the outlet temperature is 88-91° C. The air pressure is 2 bar. 105 g of a yellow-brownish powder containing Ca-D-pantothenate is obtained. The yield is 70%.

Strains utilized in the above Examples were constructed as follows:

Starting point for the development of a pantothenate production strain was Bacillus subtilis 168 (Marburg Stamm ATCC 6051), which has the genotype trpC2 (Trp⁻). From B. subtilis strain 168 the strain PY79 was generated via transduction of the Trp⁺ marker (from Bacillus subtilis wild type W23). ΔpanB and ΔpanE1 mutations were introduced into strain PY79 by classical genetic engineering (as described e.g. in Harwood, C. R. and Cutting, S. M. (editors), Molecular Biological Methods for Bacillus (1990) John Wiley & Sons, Ltd., Chichester, England).

The resulting strain was transformed with genomic DNA from Bacillus subtilis strain PA221 (genotype P₂₆panBCD, trpC2 (Trp⁻)) and genomic DNA of Bacillus subtilis strain PA303 (genotype P₂₆,panE1). The resulting strain PA327 has the genotype P₂₆panBCD, P₂₆panE1 and is auxotroph for Tryptophane (Trp⁻).

With Bacillus subtilis strain PA327 pantothenate titers of up to 3 g/L (24 h) were achieved in 10 mL cultures in SVY-Medium (25 g/L Difco Veal Infusion Broth, 5 g/L,Difco Yeast Extract, 5 g/L Na-glutamate, 2.7 g/L (NH₄)₂SO₄ dissolved in water, qs to 740 mL with water, sterilize, add 200 mL 1 M potassium phosphate, pH 7,0 and 60 mL 50% sterile Glucose-solution), which was supplemented with 5 g/L β-Alanine and 5 g/L α-ketoisovalerate.

The generation of Bacillus subtilis strain PA221 (genotype P₂₆PanBCD, trpC2 (Trp⁻)) is described in the following paragraph:

By classical genetic engineering methods the panBCD operon of Bacillus was cloned from a Bacillus subtilis GP275 Plasmid-library, using the sequence information of the panBCD operon of E. coli (see Merkel et al., FEMS Microbiol. Lett., 143, 1996:247-252).

For the cloning procedure the strain E. coli BM4062 (bir^{ls}) and the information that the Bacillus operon is located nearby the birA gene locus was used. The panBCD operon was cloned-into an E. coli replicable plasmid. To enhance the expression of the panBCD operon strong constitutive promotors of the Bacillus subtilis Phage SP01 (e.g. P₂₆) were used. In addition the ribosomal binding site (“RBS”) upstream of the panB gene was replaced by an artificial RBS having the sequence CCCTCT-AG-AAGGAGGAGAAAACATG. Just upstream of the P₂₆panBCD cassette on the plasmid a DNA fragment was inserted that is naturally located immediately upstream of the native panB gene in Bacillus. This plasmid was transformed into Bacillus subtilis strain RL-1 (by classical mutagenesis generated derivative of Bacillus subtilis 168 (Marburg strain ATCC 6051), genotype trpC2 (Trp⁻)) and via homologous recombination the native panBCD operon was replaced by the P₂₆anBCD operon. The resulting strain is called PA221 and has the genotype P₂₆panBCD, trpC2 (Trp⁻).

With Bacillus subtilis strain PA221 pantothenate titers of up to 0.92 g/L (24 h) were achieved in 10 mL cultures in SVY-Medium which was supplemented with 5 g/L β-Alanine and 5 g/L α-Ketoisovalerate.

The procedure for the preparation of Bacillus subtilis strain PA303 (genotype P₂₆panE1) is described as follows:

By knowing the E. coli panE gene sequence the Bacillus panE gene was cloned. Interestingly, in B. subtilis two genes are homologous to the E. coli panE gene, which were called panE1 and panE2. By knock out analysis it was shown, that the panE1 gene is responsible for 90% of the pantothenate production, whereas the deletion of the panE2 gene had no significant effect on pantothenate production. Also here the promotor was replaced by the strong constitutive P₂₆ promotor and the ribosomal binding site upstream of panE1 was replaced by the artificial RBS. The P₂₆EpanE1 fragment was cloned into a plasmid vector, which was designed such that the P₂₆panE1 fragment could integrate into the original native panE1 locus in the genome of Bacillus subtilis. After transformation and homologous recombination the resulting strain was called PA303, which is characterized by the genotype P₂₆panE1.

With Bacillus subtilis strain PA303 pantothenate titers of up to 1.66 g/L (24 h) were achieved in 10 mL cultures in SVY-Medium which was supplemented with 5 g/L β-Alanine and 5 g/L α-Ketoisovalerate.

The further strain development was done by transformation of PA327 with a plasmid, that contained the P₂₆ilvBNC operon and the marker gene for spectinomycin. The P₂₆ilvBNC operon integrated in the amyE locus, which was proven by PCR analysis. One of the transformants was named strain PA340 (genotype P₂₆panBCD, P₂₆panE1, P₂₆ilvBNC, specR, trpC2 (Trp⁻)).

With Bacillus subtilis strain PA340 pantothenate titers of up to 3.6 g/L (24 h) were achieved in 10 mL cultures in SVY-Medium which was supplemented with 5 g/L β-Alanine. In 10 mL cultures in:SVY-Medium which was supplemented with 5 g/L β-Alanine and 5 g/L α-Ketoisovalerate pantothenate titers of up to 4.1 g/L (24 h) were achieved.

Furtheron a deregulated ilvD cassette was introduced into strain PA340. For this reason a plasmid, which contained the ilvD gene under control of the P₂₆ promotor and the artificial RBS, was transformed into PA340. By homologous recombination the P₂₆ilvD gene was integrated into the native ilvD locus. The resulting strain PA374 has the genotype P₂₆panBCD, P₂₆panE1, P₂₆ilvBNC, P₂₆ilvD, specR and IrpC2 (Trp⁻).

With Bacillus subtilis strain PA374 pantothenate titers of up to 2.99 g/L (24 h) were achieved in 10 mL cultures in SVY-Medium which was supplemented with 5 g/L β-Alanine.

To be able to produce pantothenate without feeding the β-Alanine precursor additional copies of the aspartate-α-decarboxylase coding gene panD were introduced into strain PA374. Chromosomal DNA of the strain PA401 was transformed into PA374. By selection on tetracycline strain PA377 was obtained.

The resulting strain PA377 has the genotype P₂₆panBCD, P₂₆panE1, P₂₆ilvBNC, P₂₆ilvD, specR, tetR und trpC2 (Trp⁻).

With Bacillus subtilis strain PA377 pantothenate titers of up to 1.31 g/L (24 h) and 3.6 g/L (48 h) were achieved in 10 mL cultures in SVY-Medium without feeding of any precursor such as β-Alanine or α-Ketoisovalerate.

The generation of Bacillus subtilis strain PA401 (genotype P₂₆panD) is described in following paragraph:

The Bacillus subtilis panD gene was cloned from the panBCD operon into a plasmid vector which contains the tetracycline marker gene. Upstream of the panD gene the promotor P₂₆ and the above described artificial RBS were inserted. By restriction enzyme digest a fragment which contained the tetracycline marker gene and the P₂₆panD gene was prepared from the vector. This fragment was religated and transformed into the above described strain PA221. By doing so the fragment integrated into the genome of strain PA221. The resulting strain PA401 is characterized by the genotype P₂₆panBCD, P₂₆panD, tetR and trpC2 (Trp⁻).

With Bacillus subtilis strain PA401 pantothenate titers of up to 0.3 g/L (24 h) were achieved in 10 mL cultures in SVY-Medium which was supplemented with 5 g/L α-ketoisovalerate. In 10 mL cultures in SVY-Medium which was supplemented with 5 g/L D-pantoic acid and 10 g/L L-aspartate pantothenate titers of up to 2.2 g/L (24 h) were achieved.

Strain PA377 was transformed with chromosomal DNA of strain PY79 to generate a Tryptophan prototrophic strain. The resulting strain PA824 has the genotype P₂₆panBCD, P₂₆panE1, P₂₆ilvBNC, P₂₆ilvD, specr, tetR and Trp⁺. With Bacillus subtilis strain PA824 pantothenate titers of up to 4.9 g/L (48 h) were achieved in 10 mL cultures in SVY-Medium with no additions such as precursors.

The generation of strain PA668 is described in the following paragraph: The Bacillus panB gene was cloned from the panBCD operon and inserted into a vector plasmid, that contains the marker gene for chloramphenicol and sequences of the B. subtilis vpr locus. The strong constitutive promotor P26 was inserted upstream of the panB gene. A fragment containing the P₂₆EpanB gene, the chlorampenicol marker gene and the vpr sequence was generated by treatment with restiction enzymes. The isolated fragment was religated and used to transform strain PA824. The resulting strain was named PA668. The genotype of PA668 is P₂₆PanBCD, P₂₆panE1, P₂₆ilvBNC, P₂₆ilvD, P₂₆panB, specR, tetR and Trp⁺. Two colonies of PA668 were isolated, one of them was called PA668-2A, the other was named PA668-24.

With Bacillus subtilis strain PA668-2A pantothenate titers of up to 1.5 g/L (48 h) were achieved in 10 mL cultures in SVY-Medium with no additions such as precursors. In 10 mL cultures of SVY-Medium which was supplemented with 10 g/L L-Aspartate pantothenate titers of up to 5.0 g/L (48 h) were achieved. In 10 mL cultures in SVY-Medium which was supplemented with 5 g/L D-Pantoic acid and 10 g/L L-Aspartate pantothenate titers of up to 4.9 g/L (48 h) were achieved.

With Bacillus subtilis strain PA668-24 pantothenate titers of up to 1.8 g/L (48 h) were achieved in 10 mL cultures in SVY-Medium with no additions such as precursors. In 10 mL cultures of SVY-Medium which was supplemented with 10 g/L L-Aspartate pantothenate titers of up to 4.9 g/L (48 h) were achieved. In. 10 mL cultures in SVY-Medium which was supplemented with 5 g/L D-Pantoic acid and 10 g/L L-Aspartate pantothenate titers of up to 6.1 g/L (48 h) were achieved.

The P26 promoter seqeunce referred to herein is as follows:

gcctacctag cttccaagaa agatatccta acagcacaag
agcggaaaga tgttttgttc tacatccaga acaacctctg
ctaaaattcc tgaaaaattt tgcaaaaagt tgttgacttt
atctacaagg tgtggtataa taatcttaac aacagcagga
cgc

The above described strain PA377 was tested in a glucose-limited fermentation in SVY-Medium (25 g/L Difco Veal Infusion Broth, 5 g/L Difco Yeast Extract, 5 g/L Tryptophane, 5 g/L Na-Glutamate, 2 g/L (NH₄)₂SO₄, 10 g/L KH₂PO₄, 20 g/L K₂HPO₄, 0.1 g/L CaCl₂, 1 g/L MgSO₄, 1 g/L Na-citrate, 0.01 g/L FeSO₄*7 H₂O and 1 mL/L of a trace mineral solution (composition: 0.15 g Na₂MoO₄×2 H₂O, 2.5 g H₃BO₃, 0.7 g CoCl₂×6 H₂O, 0.25 g CuSO₄×5 H₂O, 1.6 g MnCl₂×4 H₂O, 0.3 g ZnSO₄×7 H₂O, dissolved in water, final volume 1 L)). In 10 L scale fermentations under continuous feeding of a glucose solution pantothenate titers of 18-19 g/L (36 h) and 22-25 g/L (48 h) were achieved.

The Tryptophane prototrophic derivative of PA377, PA824 was also tested in a glucose-limited fermentation in YE-Medium (10 g/L Difco Yeast Extract, 5 g/L Na-Glutamate, 8 gL (NH₄)₂SO₄, 10 g/L KH₂PO₄, 20 g/L K₂HPO₄, 0.1 g/L CaCl₂, 1 g/L MgSO₄, 1 g/L Na-citrate, 0.01 g/L FeSO₄*7 H₂O and 1 mL/L of the above mentioned trace minereal solution. In 10 L scale fermentations under continuous feeding of a glucose solution pantothenate titers of 20 g/L (36 h), 28 g/L (48 h) and 36 g/L (72 h) were achieved

PA824 was further tested in a glucose-limited fermentation in a batch media consisting of 10 g/L Difco Yeast Extract, 10 g/L NZ Amine A (Quest International GmbH, Erftstadt, Germany), 10 g/L Na-Glutamate, 4 g/L (NH₄)₂SO₄, 10 g/L KH₂PO₄, 20 g K₂HPO₄, 0.1 g/L CaCl₂, 1 g/L MgSO₄, 1 g/L Na-citrate, 0.01 g/L FeSO₄*7 H₂O and 1 mL/L of the above described trace mineral solution. In 10 L scale fermentations under continuous feeding of a glucose solution pantothenate titers of 37 g/L (36 h) and 48 g/L (48 h) were achieved.

These test fermentations exemplify strains engineered to overproduce pantothenate as well as produce pantothenate in a precursor-independent manner as defined herein.

Pantothenate titers in the fermentation broth might increase further by media optimization or development, by increasing the fermentation time, by process and strain improvement and also by the combination of the named methods. For example the abvove mentioned pantothenate titers might be achievable by fermenting strains which are derivatives of the above described strains PA824 or PA668. Derivatives can be produced by means of classical strain development such as classical mutagenesis and also by applying gene technology methodologies. By means of media, strain or process development pantothenate titers in fermentation broths can be increased to over 40, 45, 50, 55,60, 65, 70, 75, 80, 85, and >90 g/L.

Equivalents Those skilled in the art will recognize, or be able to ascertain using no more than routine experimentation, many equivalents to the specific embodiments of the invention described herein. Such equivalents are intended to be encompassed by the following claims.

What is claimed:

1. A method of producing a spray-dryable pantothenate composition, comprising culturing a pantothenate producing microorganism under Ca(OH)₂-controlled pH conditions, such that a spray-dryable pantothenate composition is produced.

2. The method of claim 1, wherein said spray-dryable pantothenate composition comprises Ca-D-pantothenate.

3. The method of claim 1, wherein the concentrated spray-dryable pantothenate composition has a dry matter content of about 10% to about 80%.

4. The method of claim 1, wherein the concentrated spray-dryable pantothenate composition has a dry matter content of about 20% to about 60%.

5. The method of claim 1, wherein the concentrated spray-dryable pantothenate composition has a dry matter content of greater than 50%.

6. The method of claim 5, wherein said pantothenate composition is further processed by spray-drying.

7. The method of claim 6, wherein said spray-dryable pantothenate composition is spray dried at an inlet temperature from about 100° C. to about 200° C.

8. The method of claim 7, wherein said spray-dryable pantothenate composition is spray dried at an outlet temperature from about 60° C. to about 100° C.

9. The method of claim 5, wherein the biomass is separated from said spray-dryable pantothenate composition prior to drying.

10. The method of claim 9, wherein the biomass is separated from the spray-dryable pantothenate composition by filtration, centrifugation, ultrafiltration, microfiltration, or combinations thereof.

11. The method of claim 1, wherein said spray-dryable pantothenate composition is concentrated.

12. The method of claim 1, wherein said Ca(OH)₂-controlled pH conditions are between about pH 6.0 and pH 11.0.

13. The method of claim 12, wherein said Ca(OH)₂-controlled pH conditions are about pH 7.0.

14. The method of claim 12, wherein said Ca(OH)₂-controlled pH conditions are about pH 10.

15. A spray-dryable pantothenate composition produced by the method of any one of claims 1-14.
